(19) United States
(12) Patent Application Publication
(10) Pub. No.: US 2025/0302385 A1
(43) Pub. Date: Oct. 2, 2025

(54) FLUID MONITORING APPARATUS AND FLUID MANAGEMENT SYSTEM FOR VENOUS SYSTEM OF HUMAN BODY

(71) Applicant: Imam Abdulrahman Bin Faisal University, Dammam (SA)

(72) Inventor: Abdulrahman Athwan S. ALQARNI, Dammam (SA)

(73) Assignee: Imam Abdulrahman Bin Faisal University, Dammam (SA)

(21) Appl. No.: 18/618,470

(22) Filed: Mar. 27, 2024

(51) Int. Cl. A61B 5/00; A61B 5/02; A61B 5/0215; A61B 5/0295; A61B 5/0537; A61B 5/0538; A61B 5/11; A61B 5/1455; A61B 5/1459

(52) U.S. Cl. CPC A61B 5/4875; A61B 5/02007; A61B 5/02042; A61B 5/02158; A61B 5/0295; A61B 5/0537; A61B 5/0538; A61B 5/1116; A61B 5/14551; A61B 5/1459; A61B 5/6852; A61B 2560/0462; A61B 2562/0219; A61B 2562/0247; A61B 2562/0261; A61B 2562/063; A61B 2562/227

(57) ABSTRACT

A fluid management system and a fluid monitoring apparatus for invasive monitoring of a venous system of a human body includes a catheter tube, having a lumen, insertable into a blood vessel of the venous system of the human body and a controller communicatively coupled to four sensors attached to an internal surface of the catheter tube for contacting with the fluid. The four sensors includes a first pressure sensor attached at an insertable tip of the catheter tube, a fluid movement sensor attached proximal to the insertable tip, a vasodilation detector and a second pressure sensor attached at a middle section of the catheter tube, and a volume sensor attached at a distal section of the catheter tube.

### BACKGROUND

### Technical Field

The present disclosure is directed to a method, apparatus and system for fluid monitoring in a venous system of a human body, and more particularly, directed to a fluid monitoring apparatus and a fluid management system for the venous system of the human body.

### Description of Related Art

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Fluid management of human bodies in healthcare facilities is a critical aspect of patient care, encompassing a balance between maintaining optimal hydration and preventing fluid overload. Appropriate fluid management impacts multiple physiological functions, such as, but are not limited to, maintaining blood pressure and tissue perfusion to support appropriate organ function. However, reliance on traditional methods and subjective observations for fluid management may lead to challenges in accurately assessing and addressing fluid dynamics of the human body and the fluid needs of a patient.

Presently, fluid management relies on personal judgment of healthcare practitioners, visual observations, and general guidelines. Healthcare practitioners gauge a patient's fluid needs based on factors like urine output, clinical appearance, and experience. While these methods may have been adequate to some extent, they are fraught with limitations, particularly in the face of complex physiological interactions and variations among individual patients. The complexity of fluid dynamics within the human body is multifaceted, as fluid balance is a dynamic process influenced by factors such as varying metabolic rates, surgical interventions, medications, and underlying medical conditions. These variables contribute to fluid shifts that may be difficult to quantify and manage accurately through traditional approaches. Further, each patient is unique, with distinct physiological responses to fluid administration. Personalized fluid needs depend on factors including, age, weight, medical history, organ function, and the like. Traditional methods struggle to account for the above-mentioned individual variability. Furthermore, fluid management is intricately linked to various physiological systems, including the cardiovascular system, renal system, and respiratory system. Fluid shifts in one system may impact others, leading to cascading effects that may require precise monitoring. Additionally, fluid losses and gains are not limited to urinary output. Insensible losses through respiration and perspiration, as well as gastrointestinal losses, contribute to the overall fluid balance. Moreover, fluid overload and dehydration may manifest differently in patients, making diagnosis based solely on visual cues and personal judgment challenging. Over-reliance on traditional methods of fluid management may result in inadequate or excessive fluid administration.

U.S. Pat. No. 8,603,000 describes a conductance catheter for measuring the volume of a fluid. The conductance catheter includes a series of electrodes and a circuit to compensate for variations in sensitivity of the electrodes in the catheter. Further, a resistivity sensor is provided for determining the resistivity of a fluid. The resistivity sensor includes a series of electrodes spaced such that the total distance between endmost electrodes does not exceed the diameter of the catheter deploying the sensor. However, U.S. Pat. No. 8,603,000 does not describe real-time and dynamic fluid monitoring.

U.S. Pat. No. 11,559,257 describes a device including a catheter insert elongated body defining a body lumen. The catheter insert elongated body is configured to be at least partially inserted to a catheter lumen defined by a catheter without covering a first fluid opening of the catheter and to form a fluidically tight coupling with the catheter, and one or more sensors positioned on the elongated body. At least one of the sensors is configured to sense a substance of interest. The catheter insert elongated body includes a material that is a substantially non-permeable to the substance of interest. However, U.S. Pat. No. 11,559,257 does not specifically describe invasive monitoring of a plurality of parameters included in the fluid dynamics of human body.

Accordingly, it is one object of the present disclosure is to provide a fluid monitoring apparatus and a fluid management system, that may circumvent the aforementioned drawbacks of conventional fluid monitoring systems such as inability to monitor the plurality of parameters included in the fluid dynamics of the human body and inadequately catering to individual needs of patients.

### SUMMARY

In an exemplary embodiment, a fluid monitoring apparatus for a venous system of a human body is described. The fluid monitoring apparatus includes a catheter tube insertable into a blood vessel of the venous system of the human body to have fluid from the blood vessel enter and exit the catheter tube. The catheter tube includes at least one lumen. The fluid monitoring apparatus further includes at least four sensors attached to an internal surface of the catheter tube and in contact with the fluid when inserted in the blood vessel. The at least four sensors are selected from a first pressure sensor attached at an insertable tip of the catheter tube, a fluid movement sensor attached proximal to the insertable tip of the catheter tube, a vasodilation detector attached at a first sector of a middle section of the catheter tube, a second pressure sensor attached at a second sector of the middle section of the catheter tube, and a volume sensor attached at a distal section of the catheter tube. The fluid monitoring apparatus further includes a controller communicatively coupled to the at least four sensors.

In some embodiments, the controller further includes a transducer in communication with the first pressure sensor and the second pressure sensor.

In some embodiments, each of the first pressure sensor and the second pressure sensor are at least one of a piezo-resistive pressure sensor, photo-electric sensor, and a photo-optic sensor. The fluid movement sensor is at least one of an electromagnetic sensor and an electrochemical sensor. The vasodilation detector is at least one of a capacitive strain gauge sensor or a bio-impedance sensor, and the volume sensor is at least one of a photoplethysmography (PPG) sensor and an electromagnetic sensor.

In some embodiments, the controller is in communication with an external position sensor.

In some embodiments, the external position sensor is configured to be connected to an external surface of the human body.

In some embodiments, the fluid monitoring apparatus further includes an external cable connected between the external position sensor and the controller.

In some embodiments, the fluid monitoring apparatus further includes a plurality of cables connected between the at least four sensors attached to the catheter tube and the controller.

In some embodiments, each of the at least four sensors has a length in a range from 1 centimeters (cm) up to 3 cm.

In some embodiments, each of the at least four sensors has an external diameter in a range from 1 millimeters (mm) up to 2 mm.

In some embodiments, each of the at least four sensors has an external diameter up to 0.9 times of a diameter of the catheter tube.

In some embodiments, the at least four sensors are longitudinally spaced apart from one another inside the catheter tube, and a flexible separator is present between each sensor of the at least four sensors. The flexible separator includes a flexible cellular polymer.

In some embodiments, the catheter tube is made of a flexible material.

In some embodiments, the catheter tube includes an insertion port having a length of at least 1 cm at the insertable tip of the catheter tube for insertion of a guidewire from a center point of the insertion port and along an edge of the at least four sensors attached to the catheter tube.

In some embodiments, the catheter tube is a central venous catheter.

In some embodiments, the catheter tube is integrated into an intra-aortic balloon pump.

In another exemplary embodiment, a fluid management system for an invasive monitoring of a venous system of a human body is described. The system includes a catheter tube insertable into a blood vessel of the venous system of the human body through a guidewire. The catheter tube includes at least one lumen. Fluid from the blood vessel enters and exits the catheter tube. The fluid management system further includes at least four sensors attached to an internal surface of the catheter tube and in contact with the fluid when inserted in the blood vessel. The at least four sensors are selected from a first pressure sensor at an insertable tip of the catheter tube configured to measure a pressure at the insertable tip of the catheter tube, a fluid movement sensor proximal to the insertable tip of the catheter tube configured to measure a fluid movement rate of the fluid in contact with the fluid movement sensor, a vasodilation detector at a first sector of a middle section of the catheter tube configured to measure a change in a diameter of the blood vessel for an assessment of at least one of a vasodilation or a vasoconstriction, and a volume sensor at a distal section of the catheter tube configured to measure a change in a volume of the fluid in contact with the volume sensor. The fluid management system includes a controller communicatively coupled to the at least four sensors configured to receive a measurement value from each of the at least four sensors and to assess a fluid requirement of the human body. The fluid management system further includes a plurality of cables extending from the catheter tube to the controller configured to conduct communication between the at least four sensors and the controller. An insertion port having a length of at least 1 cm at the insertable tip of the catheter tube for insertion of the guidewire from a center point of the insertion port and along an edge of the at least four sensors attached to the catheter tube.

In some embodiment, the controller further includes a transducer in communication with the first pressure sensor.

In some embodiments, the fluid management system further includes a second pressure sensor attached at a second sector of the middle section of the catheter tube and configured to measure and communicate a pressure of the fluid against a wall of the blood vessel to the controller.

In some embodiments, the controller is connected to an external position sensor through an external cable.

In some embodiments, the external position sensor is configured to be connected to an external surface of the human body and to detect and communicate a change in an orientation of the human body to the controller.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof may be readily obtained as the same becomes better understood by reference to the following detailed description when considered in connection with the accompanying drawings, wherein:

FIG. 1 is a schematic diagram showing a fluid monitoring apparatus attached to a human body, according to certain embodiments.

FIG. 2A is a schematic diagram of a catheter tube of the fluid monitoring apparatus, according to certain embodiments.

FIG. 2B is a schematic enlarged view of a tip portion of the catheter tube showing multiple sensors disposed therein, according to certain embodiments.

FIG. 2C is a schematic diagram showing an intra-aortic balloon pump integrated with the catheter tube, according to certain embodiments.

FIG. 3 is a schematic block diagram of a fluid management system, according to certain embodiments.

FIG. 4 is a schematic diagram of an exemplary working environment of the fluid monitoring apparatus and the fluid management system, according to certain embodiments.

FIG. 5 is an illustration of a non-limiting example of details of a computing hardware used in the fluid management system, according to certain embodiments.

FIG. 6 is an exemplary schematic diagram of a data processing system used within the fluid management system, according to certain embodiments.

FIG. 7 is an exemplary schematic diagram of a processor used with the fluid management system, according to certain embodiments.

FIG. 8 is an illustration of a non-limiting example of distributed components which may share processing with a controller of the fluid management system, according to certain embodiments.

### DETAILED DESCRIPTION

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed towards a fluid management system and a fluid monitoring apparatus for maintaining optimum fluid levels in a human body. The fluid monitoring apparatus includes a catheter tube having a plurality of sensors in order to monitor fluid levels inside the human body, invasively. The plurality of sensors is communicatively coupled with a controller in order to provide master control of the apparatus to a healthcare practitioner. The fluid monitoring apparatus, as incorporated in the fluid management system, may provide precise control over fluid levels inside a body of a patient. The invasive nature of the fluid management system enables the healthcare practitioner to make precise adjustments in a set of inputs transmitted to the fluid monitoring apparatus.

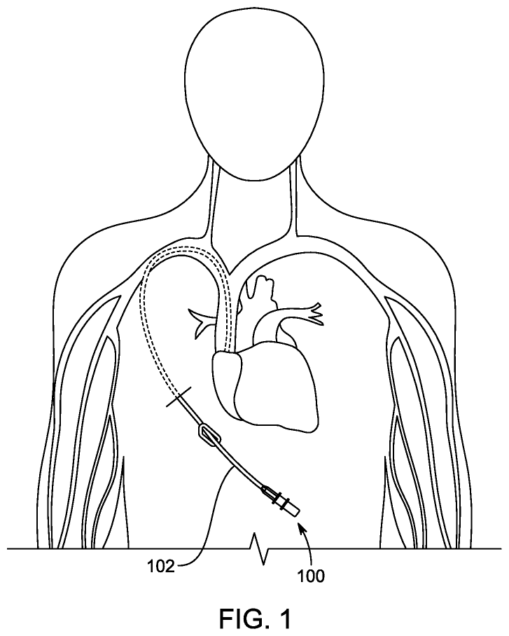

Referring to FIG. 1, a schematic diagram of a fluid monitoring apparatus 100 attached to a human body is illustrated, according to certain embodiments. The fluid monitoring apparatus 100 is alternatively referred to as ‘the apparatus 100’ hereinafter, for brevity in explanation. The apparatus 100 includes a catheter tube 102 insertable into a blood vessel of a venous system of the human body to have fluid from the blood vessel enter and exit the catheter tube 102. In particular, FIG. 1 depicts a referential representation of the catheter tube 102 inserted into the blood vessel of the human body. In some embodiments, a type of the catheter tube 102 may include, but is not limited to, foley catheter, intermittent catheter, suprapubic catheter, and central venous catheter. In an embodiment of the present disclosure, the catheter tube 102 is a central venous catheter and made of a flexible material. In general, central venous catheter is a flexible tube-like device that helps in transmitting a plurality of drugs and treatment protocols, intravenously, for various medical conditions, directly into blood vessels in which it is administered.

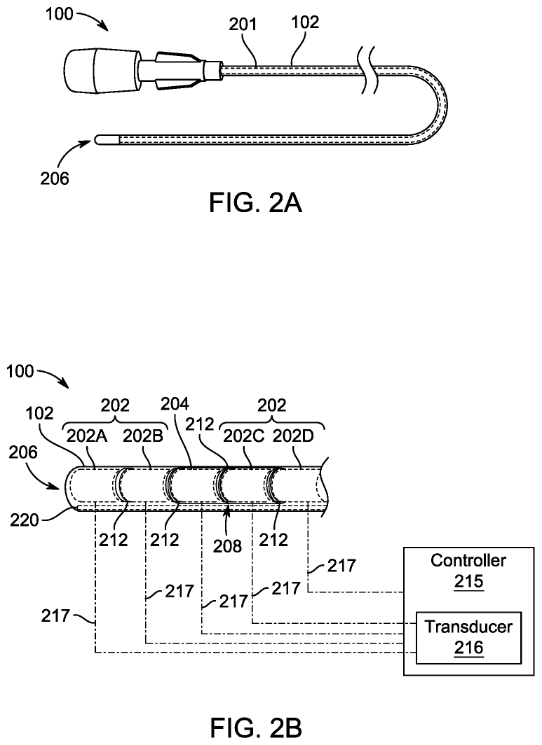

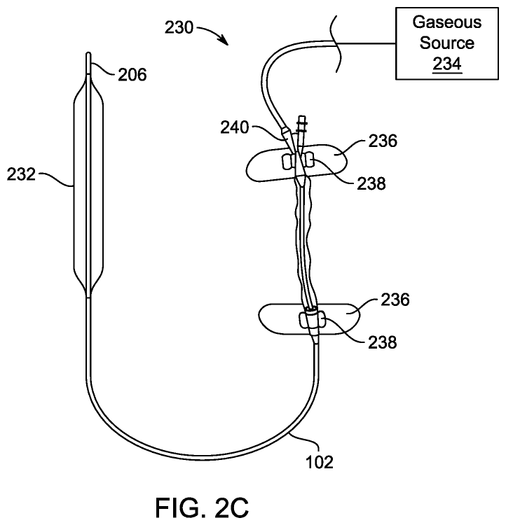

Referring to FIG. 2A, a schematic diagram of the catheter tube 102 is illustrated, according to certain embodiments. In particular, the catheter tube 102 is made of the flexible material such as, but is not limited to, polyurethane, silicone rubber, polyethermide, polypropylene, and polycarbonate. The catheter tube 102 is sterilized prior to administering in the blood vessel of the human body. In an embodiment of the present disclosure, the catheter tube 102 includes at least one lumen 201. The lumen 201 refers to an internal passage defined in the catheter tube 102. In some embodiments, the catheter tube 102 may have more than one lumen 201 based on an application of the catheter tube 102 and depending on a requirement of a patient.

Referring to FIG. 2B, a schematic enlarged view of a tip portion of the catheter tube 102 is illustrated, according to certain embodiments. The apparatus 100 includes at least four sensors 202 attached to an internal surface of the catheter tube 102 and preferably at least partially in contact, e.g., physically (fluidly), thermally or electromagnetically, with the fluid when inserted in the blood vessel of the human body. The internal surface of the catheter tube 102 is defined by the lumen 201 thereof. In some embodiments, the at least four sensors 202 are selected from a first pressure sensor 202A, a fluid movement sensor 202B, a second pressure sensor 202C, and a volume sensor 202D. The at least four sensors are preferably arranged in tandem with no or minimal spacing between sensors, e.g., no more than 0.1 to 5 mm, preferably 0.5 to 2 mm spacing between sensors. The at least at four sensors are preferably at least partially, preferably majorly, disposed within the lumen 102 or defining an inner wall of the lumen. Preferably the at least four sensors are disposed in the lumen such that each sensor occupies a distinct cylindrical portion of the lumen tip with no overlap with the portions of the lumen tip that are occupied by other sensors.

The apparatus 100 further includes a vasodilation detector 204 preferably disposed outside the lumen or at a location external to the at least four sensors, e.g., at or outside of an outer wall of the lumen.

In some embodiments, the first pressure sensor 202A is attached at an insertable tip 206 of the catheter tube 102 and the fluid movement sensor 202B is attached proximal to the insertable tip 206 of the catheter tube 102. Further, the vasodilation detector 204 is preferably attached at a first sector of a middle section 208 of the catheter tube 102 and the second pressure sensor 202C is attached at a second sector of the middle section 208 of the catheter tube 102. Furthermore, the volume sensor 202D is attached at a distal section of the catheter tube 102.

In some embodiments, each of the first pressure sensor 202A and the second pressure sensor 202C are at least one of a piezo-resistive sensor, photo-electric sensor, and a photo-optic sensor. In general, piezo-resistive sensors or piezo-resistive strain gauges are a type of pressure sensors. Piezo-resistive sensors use a change in electrical resistance of a material when stretched to measure the pressure. In particular, piezo-resistive sensors use a strain gauge made from a conductive material that changes its electrical resistance when it is stretched. The strain gauge may be attached to a diaphragm that recognizes a change in resistance when the sensor element is deformed and the change in resistance is converted to an output signal. Further, photo-electric sensor and photo-optic sensor are type of sensors that detect the presence, absence, distance, and intensity of a component via light energy. In some embodiments, the fluid movement sensor 202B is at least one of an electromagnetic sensor and an electrochemical sensor. In general, electromagnetic sensors are solid state devices used in detecting and sensing distance, speed, rotation, angle, and position by converting magnetic information into electrical signal. The electrochemical sensors convert information pertaining to electrochemical reactions in a fluid to readable analytical signals. In an embodiment of the present disclosure, the fluid movement sensor 202B is a Doppler ultrasound sensor.

In some embodiments, the volume sensor 202D is at least one of a photoplethysmography (PPG) sensor and an electromagnetic sensor. In general, PPG sensor obtains a plethysmogram that may be used to detect blood volume changes in microvascular bed of tissue. In an example, a pulse oximeter is a PPG sensor. Moreover, in some embodiments, the vasodilation detector 204 is at least one of a capacitive strain gauge sensor or a bio-impedance sensor. In general, bio-impedance sensors are used to detect biological components and molecules by measuring impedance changes. Bio-impedance sensors apply sinusoidal voltage to specific frequencies and measure electrical impedance with alternating current flow.

In some embodiments, each of the at least four sensors 202 has an external diameter up to 0.9 times of a diameter of the catheter tube 102. In other words, the at least four sensors 202 and the vasodilation detector 204 are preferably smaller in diameter than an inner diameter of the catheter tube 102 in order to fit inside the catheter tube 102 (though minor portions of the sensors may protrude outside or be disposed at the outer surface of the catheter). In particular, each of the at least four sensors 202 has an external diameter in a range from 1 millimeter (mm) up to 2 mm and each of the at least four sensors 202 has a length in a range of 1 centimeter (cm) up to 3 cm. However, in some embodiments, the dimensional specifications of the at least four sensors 202 may be defined in order to better suit pediatric use case scenarios. Consequently, the catheter tube 102 can be configured to have a smaller diameter in order to fit relatively narrower blood vessels of a pediatric patient. Furthermore, in some embodiments, the at least four sensors 202 are longitudinally spaced apart from one another inside the catheter tube 102 by a flexible separator 212. In other words, the flexible separator 212 is present between each sensor of the at least four sensors 202, as such, the flexible separator 212 provides cushioning and damping to each sensor of the at least four sensors 202, when a force is applied by the fluid present in the blood vessel. In some embodiments, the flexible separator 212 is made of a flexible cellular polymer.

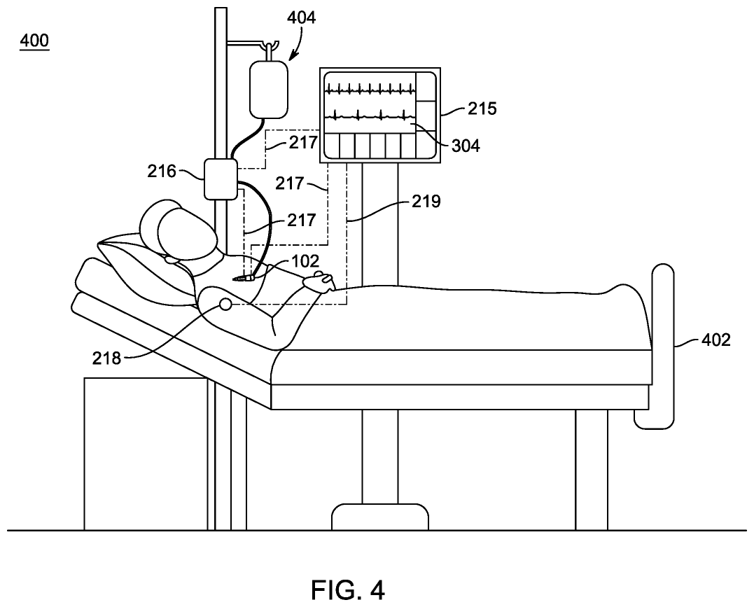

In some embodiments, the apparatus 100 further includes a controller 215. In particular, the controller 215 is communicatively coupled to the at least four sensors 202 and the controller 215 further includes a transducer 216, in communication with the first pressure sensor 202A and the second pressure sensor 202C. The controller 215 is configured to collect a first set of data from the at least four sensors 202. The first set of data includes a measurement value of a plurality of factors including, but not limited to, pressure and volume of the fluid flowing through the catheter tube 102. Further, the apparatus 100 includes a plurality of cables 217 connected between the at least four sensors 202 attached to the catheter tube 102 and the controller 215. As such, the controller 215 is electrically and communicatively coupled to the catheter tube 102 via the plurality of cables 217. In some embodiments, the plurality of cables 217 may be secured into a single cable casing to ensure entanglement free operation of the apparatus 100. The plurality of cables 217 includes at least one of an insulated copper wire, insulated aluminum wire, insulated inert metal wire, or a combination thereof. In some embodiments, the controller 215 is configured to be in communication with an external position sensor 218. In an embodiment of the present disclosure, the external position sensor 218 is configured to be connected to an external surface of the human body, as shown in FIG. 4. As such, the external position sensor 218 is electrically coupled to the controller 215 via an external cable 219, shown in FIG. 4.

In some embodiments, the catheter tube 102 includes an insertion port 220 having a length of at least 1 cm defined at the insertable tip 206 of the catheter tube 102. The insertion port 220 is configured for an insertion of a guidewire from a center point of the insertion port 220 and along an edge of the at least four sensors 202 attached to the catheter tube 102. In some embodiments, the guidewire may run along an entirety of the catheter tube 102, as such, the guidewire facilitates the insertion of the catheter tube 102 into the blood vessels of the patient. In general, a guidewire is a device used to guide the catheter tube 102 into place during central venous insertions. The purpose of the guidewire is to gain access to the blood vessels using a minimally invasive technique. The guidewire inserted through the insertion port 220 enables the healthcare practitioner to insert the apparatus 100 into the blood vessels of the patient, precisely and in a relatively painless manner. Further, a curvature feature of the insertable tip 206 may be designed to reduce pain during insertion process of the catheter tube 102 into the blood vessels.

Referring to FIG. 2C, a schematic diagram of the catheter tube 102 integrated with an intra-aortic balloon pump 230 is illustrated, according to certain embodiments. In particular, an implementation of the catheter tube 102 of the present disclosure with the intra-aortic balloon pump 230 is shown in FIG. 2C. In general, an intra-aortic balloon pump (IABP) is a mechanical device that increases myocardial oxygen perfusion and indirectly increases cardiac output through afterload reduction. A balloon membrane 232 is disposed at a proximal end of the catheter tube 102, near the insertable tip 206 and an inner lumen is defined along a length of the catheter tube 102. A distal end of the catheter tube 102 may be fluidly coupled with a gaseous source 234 to inflate or deflate the balloon membrane 232. In an example, the balloon membrane 232 may sit in aorta, approximately 2 cm from left subclavian artery. Further, the balloon membrane 232 may inflate and deflate via counter pulsation, as such, the balloon membrane 232 actively deflates in systole and inflates in diastole. In general, systole refers to a phase of heartbeat when heart muscles contracts and pumps blood from heart chambers into a plurality of arteries of the human body and diastole refers to a phase when the heart muscles are relaxed, and the chambers of the heart are refilling with blood. Systolic deflation decreases afterload through a vacuum effect and indirectly increases forward flow from heart. Furthermore, diastolic inflation increases blood flow to coronary arteries via retrograde flow. These above mentioned processes work in conjunction to decrease myocardial oxygen demand and increase myocardial oxygen supply. In some embodiments, a pair of seal 236 and a pair of suture pads 238 are configured to securely hold the catheter tube 102 onto a site of insertion of the catheter tube 102 into the blood vessels. Furthermore, a Y-fitting 240 enables the catheter tube 102 to be supplied with one or more fluids during a treatment of the patient.

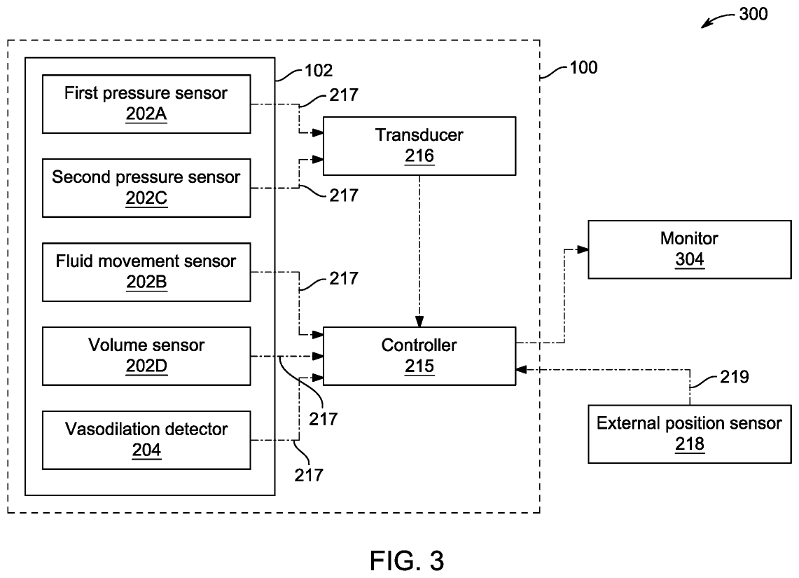

Referring to FIG. 3, a schematic block diagram of a fluid management system 300 for an invasive monitoring of the venous system of the human body is illustrated, according to certain embodiments. The fluid management system 300 is alternatively referred to as ‘the system 300’, hereinafter, for brevity in explanation. In particular, the system 300 enables the healthcare practitioner to monitor and manage the fluid levels and associated parameters, of the patient, in real-time. The system 300 further provides the ability to the healthcare practitioner to dynamically adjust a plurality of factors affecting the above mentioned fluid levels. The system 300 includes the catheter tube 102, the controller 215, the transducer 216, the plurality of cables 217, the external position sensor 218, and a monitor 304. In some embodiments, the system 300 includes the catheter tube 102 insertable into the blood vessel of the venous system of the human body through the guidewire. The catheter tube 102 includes the at least one lumen 201 configured in such a way that the fluid from the blood vessel enters and exits the catheter tube 102. The system 300 includes the at least four sensors 202 attached to the internal surface of the catheter tube 102. The at least four sensors 202 are in contact with the fluid when inserted in the blood vessel.

The at least four sensors 202 are selected from the first pressure sensor 202A, the fluid movement sensor 202B, the second pressure sensor 202C, and the volume sensor 202D. The system 300 further includes the vasodilation detector 204. The first pressure sensor 202A is positioned at the insertable tip 206 of the catheter tube 102. In some embodiments, the first pressure sensor 202A is configured to measure a pressure at the insertable tip 206 of the catheter tube 102, as such, the first pressure sensor 202A provides a baseline pressure reference. The baseline pressure may be referred in future to adjust and amend a plurality of factors such as, fluid infusion rate, medication infusion rate, and the like. In particular, the first pressure sensor 202A measures blood pressure which correlates to a force exerted by the fluids present in the blood vessels. The first pressure sensor 202A provides real-time measurements of the force exerted by circulating blood against walls of the blood vessels. Further, the first pressure sensor 202A facilitates the apparatus 100 of the system 300 to track changes in blood pressure, such as increment or decrement in the blood pressure, which may be indicative of at least one of, fluid shifts, changes in cardiac output, and other physiological responses. In an aspect, the first pressure sensor 202A allows the apparatus 100 and the system 300 to differentiate between blood pressure changes due to fluid loss and blood pressure changes due to other physiological factors. Further, the system 300 includes the second pressure sensor 202C, attached at the second sector of the middle section 208 of the catheter tube 102. The second pressure sensor 202C is configured to measure and communicate the pressure of the fluid against a wall of the blood vessel to the controller 215. The first and the second pressure sensors 202A, 202C are designed to be minimally invasive and fit within the catheter tube 102.

The fluid movement sensor 202B is positioned proximal to the insertable tip 206 of the catheter tube 102. In some embodiments, the fluid movement sensor 202B is configured to measure a fluid movement rate of the fluid in contact with the fluid movement sensor 202B. In other words, the fluid movement sensor 202B capture fluid movement rates as fluids flow past the fluid movement sensor 202B. The proximal positioning of the fluid movement sensor 202B provides accurate readings and minimizes potential disturbances that may be caused due to the insertion port 220 of the apparatus 100. In an example, the fluid movement sensor 202B may be an impedance-based sensor having relatively compact dimensions. In another example, the fluid movement sensor 202B may be a Doppler ultrasound sensor having slightly larger dimensions due to the need of accommodating a device for emitting and receiving ultrasound waves. In an embodiment, the fluid movement sensor 202B, positioned proximal to the insertable tip 206, assesses the rate at which fluids are moving within the blood vessels. The fluid movement sensor 202B may provide an insight into overall movement of fluids with regards to a plurality of parameters including, but not limited to, fluid intake, urinary output, insensible losses, and metabolic demands. In another embodiment, the fluid movement sensor 202B allows the system 300 to keep track of fluid transport mechanisms present in the body and subsequently, calculate an efficiency rating for fluid distribution in the body. The efficiency rating may be calculated by comparing real-time fluid movement sensor 202B reading with a predetermined standard fluid movement rate.

The volume sensor 202D is positioned at the distal section of the catheter tube 102. In particular, the volume sensor 202D is configured to measure a change in volume of the fluid in contact with the volume sensor 202D, as such, the volume sensor 202D provides information about net gain or net loss of the fluids over a defined period. The volume sensor 202D is vital for assessment of changes in a localized area, such as, but not limited to, vascular system, and a plurality of specific body cavities. The positioning of the volume sensor 202D at the distal section allows the volume sensor 202D to record a magnitude of change in volume of the fluid, as the fluid passes through the catheter tube 102. The volume sensor 202D monitors and quantify accumulation or depletion of the fluids in the above mentioned localized area and tracks fluid shifts in response to medical interventions such as fluid administration and fluid drainage. In some embodiments, the volume sensor 202D may be an impedance based sensor and may have slightly larger dimensional specifications in order to accommodate electrodes and circuitry for measuring electrical impedance changes.

The vasodilation detector 204 is positioned at the first sector of the middle section 208 of the catheter tube 102. The vasodilation detector 204 is configured to measure a change in diameter of the blood vessel for an assessment of at least one of a vasodilation or a vasoconstriction. In general, vasodilation refers to an increase in diameter (expansion) of the blood vessels whereas vasoconstriction refers to a decrease in diameter (shrinkage) of the blood vessels. In an aspect, the vasodilation detector 204 measures a vascular tone of the body. In general, vascular tone refers to a degree of vasoconstriction experienced by blood vessel relative to its maximum vasodilated state. The changes in the diameter of the blood vessels affect peripheral resistance present in the blood vessels and may impact blood pressure regulation. The vasodilation detector 204 may detect the above mentioned peripheral resistance change and subsequently report the data as recorded. Further, the vasodilation detector 204 assesses how the blood vessels are responding to changes in physiological state of the body. Physiological state may refer to a shift in fluid balance or regulatory mechanisms. Further, the vasodilation detector 204 facilitates in differentiating between vasodilation caused by blood pressure changes and vasodilation caused by fluid loss. A set of data from vasodilation detector 204 may contribute towards an accurate assessment of fluid balance and blood pressure changes of the human body, with reference to the vascular tone of the body. In order to summarize, the vasodilation detector 204 primarily focuses on monitoring changes in the diameter of the blood vessels.

Further, referring to FIG. 2B and FIG. 3, the first set of data having the measurement values from the at least four sensors 202 is used to quantify a fluid requirement of the human body. In other words, the controller 215 receives the measurement value from each of the at least four sensors 202 to assess the fluid requirement of the human body. Furthermore, the plurality of cables 217 extending from the catheter tube 102 to the controller 215 are configured to conduct communication between the at least four sensors 202 and the controller 215. Moreover, the controller 215 included in the system 300 is configured to couple with the transducer 216. In general, transducer converts energy from one form to another and a signal in one form of energy to a signal in another form of energy. Transducers are employed at the boundaries of automation, measurement, and control systems, where electrical signals are converted to and from other physical quantities such as, energy, force, torque, light, motion, position, and the like. The process of converting one form of energy to another is known as transduction.

In some embodiments, the controller 215 is in communication with the external position sensor 218. Further, as can be seen from FIG. 4, the external position sensor 218 is configured to be connected to the external surface of the human body. The apparatus 100 and the system 300 further includes the external cable 219 connected between the external position sensor 218 and the controller 215. As such, the controller 215 is connected to the external position sensor 218 through the external cable 219. In other words, the external cable 219 communicatively and electrically couples the external position sensor 218 with the controller 215. In particular, the external position sensor 218 is configured to be connected to the external surface of the human body and to detect and communicate a change in an orientation of the human body to the controller 215. As such, the external position sensor 218 senses a change in position of the human body, and reports, a data related to the change in position, to the controller 215 via the external cable 219. In general, position changes such as, trendelenburg position and reverse trendelenburg position, affect the blood pressure of human bodies. Trendelenburg position refers to a state where feet of a particular patient are elevated higher than head of the particular patient, and reverse trendelenburg position refers to a state where the head of the particular patient is elevated higher than the feet of the particular patient.

In some embodiments, the monitor 304 displays a plurality of readings, accumulated and sourced from the apparatus 100 and the system 300 in a synergistic manner. The monitor 304 enables the healthcare practitioner to visually monitor and precisely finetune the plurality of factors and parameters affecting the fluid dynamics of the human body. In some embodiments, the monitor 304 may include a digital display. The digital display may include a plurality of display technologies such as, but are not limited to, an LCD display, a light emitting diode display, a cathode ray display. Further, the digital display may be a touch sensitive panel. In an aspect, the monitor 304 may display a first blood pressure measurement from the first pressure sensor 202A, a fluid movement rate from the fluid movement sensor 202B, a second blood pressure measurement from the second pressure sensor 202C, localized changes in vascular tone from the volume sensor 202D, and a real-time diameter of the blood vessel from the vasodilation detector 204. The aforementioned values are compared against a set of standard or optimal values and then changes are made, by the healthcare practitioner in real-time, to adjust the values received from the apparatus 100 and the system 300 to match the optimal values. These changes may include, but are not limited to, a change in fluid volume being administered, a positional change, a change in medications being administered, or a combination thereof.

Referring to FIG. 4, an exemplary working environment 400 of the apparatus 100 and the system 300 is illustrated, according to certain embodiments. In the exemplary working environment 400, a patient lies down on an adjustable bed 402 and is provided medication through a fluid drip 404. In particular, the fluid drip 404 may include a medicine, a fluid, or a combination thereof. A flow rate of the fluid flowing through the fluid drip 404 may be governed by the system 300 based on the inputs of the healthcare practitioner via the controller 215. In an example, the system 300 may be used to detect changes in venous pressure during Trendelenburg positioning of the patient. In such a case, the first pressure sensor 202A may detect an abrupt increment in magnitude of venous pressure. The fluid movement sensor 202B may register an increase in blood flow rate. The second pressure sensor 202C may register a relatively constant arterial pressure. The volume sensor 202D may register a transient increase in blood volume. The vasodilation detector 204 may register a constant vascular due to the position change. Further, a heart rate monitor may be included to monitor a heart rate of the patient. In another example, the system 300 may be used to detect fluid overload due to excess fluid administration. In such a case, the first pressure sensor 202A may detect a gradual increment in the magnitude of the venous pressure. The fluid movement sensor 202B may register an increase in the blood flow rate of the patient. The second pressure sensor 202C may register a constant arterial pressure of the patient. The volume sensor 202D may register a steady increase in the blood volume. The vasodilation detector 204 may register an increase in vascular width due to increased fluid volume. The heart rate monitor may register a constant heart rate. In yet another example, the system 300 may be used to detect hemorrhage and decreased blood volume. In general, hemorrhage may be defined as a loss of blood volume from a blood vessel due to an internal or an external injury or certain diseases. In such a case, the first pressure sensor 202A may register a decrease in venous pressure of the patient. The fluid movement sensor 202B may register a decrease in blood flow due to reduced blood volume of the patient. The second pressure sensor 202C may register an increase in arterial pressure in response to decreased blood volume of the patient. The volume sensor 202D may register a transient decrease in blood volume of the patient. The vasodilation detector 204 may register a decrease in vascular width due to reduced blood volume. Furthermore, the heart rate monitor may register an increase in the heart rate of the patient, as a compensatory response against loss of blood volume.

In yet another example of the present disclosure, the system 300 may be integrated with a fluid delivery machine. The fluid delivery machine may be based on an equation that predicts a fluid need of the patient. The equation may be used to estimate a tailored amount of fluid for multiple patients depending on their unique physiological responses. The equation may calculate a specific amount of fluid based on fluid requirements of the patient, and the fluid delivery machine, assisted by the system 300, may be adjusted to deliver the specific amount of fluid precisely. The equation may be hypothesized as follows,

Specific amount of fluid=ΔV+(k×ΔP×Δt)−(m×ΔR)+(n×Q),

where, ΔV is change in fluid volume, ΔP is change in blood pressure, Δt is change in time, ΔR is change in vascular resistance, and Q is fluid movement rate. Further, k, m, and n are coefficients.

The aforementioned parameters may be measured for individual patients and subsequently the equation may predict desired and accurate amount of fluids to be administered to each of the individual patients. Thus, the fluid delivery machine assisted by the system 300 automatically judges and adjusts the fluid needs for the individual patients.

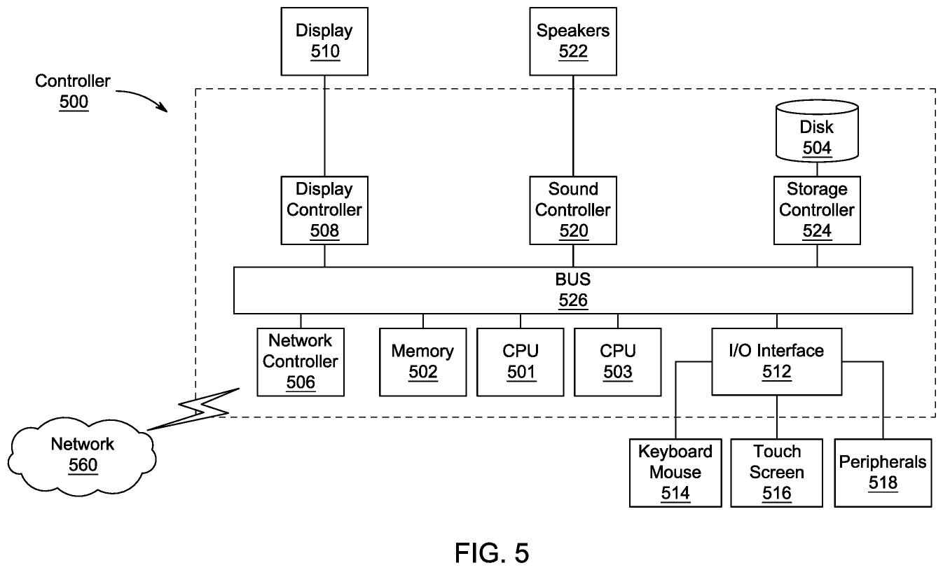

Next, further details of the hardware description of the computing environment according to exemplary embodiments is described with reference to FIG. 5. In FIG. 5, a controller 500 described is representative of the system 300 of FIG. 3 in which the controller is a computing device which includes a CPU 501 which performs the processes described above/below. The process data and instructions may be stored in memory 502. These processes and instructions may also be stored on a storage medium disk 504 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 501, 503 and an operating system such as Microsoft Windows 7, Microsoft Windows 10, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, CPU 501 or CPU 503 may be a Xenon or Core processor from Intel of America or an Opteron processor from AMD of America, or may be other processor types that would be recognized by one of ordinary skill in the art. Alternatively, the CPU 501, 503 may be implemented on an FPGA, ASIC, PLD or using discrete logic circuits, as one of ordinary skill in the art would recognize. Further, CPU 501, 503 may be implemented as multiple processors cooperatively working in parallel to perform the instructions of the inventive processes described above.

The computing device in FIG. 5 also includes a network controller 506, such as an Intel Ethernet PRO network interface card from Intel Corporation of America, for interfacing with network 560. As can be appreciated, the network 560 can be a public network, such as the Internet, or a private network such as an LAN or WAN network, or any combination thereof and can also include PSTN or ISDN sub-networks. The network 560 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The computing device further includes a display controller 508, such as a NVIDIA GeForce GTX or Quadro graphics adaptor from NVIDIA Corporation of America for interfacing with display 510, such as a Hewlett Packard HPL2445w LCD monitor. A general purpose I/O interface 512 interfaces with a keyboard and/or mouse 514 as well as a touch screen panel 516 on or separate from display 510. General purpose I/O interface also connects to a variety of peripherals 518 including printers and scanners, such as an OfficeJet or DeskJet from Hewlett Packard.

A sound controller 520 is also provided in the computing device such as Sound Blaster X-Fi Titanium from Creative, to interface with speakers/microphone 522 thereby providing sounds and/or music.

The general purpose storage controller 524 connects the storage medium disk 504 with communication bus 526, which may be an ISA, EISA, VESA, PCI, or similar, for interconnecting all of the components of the computing device. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

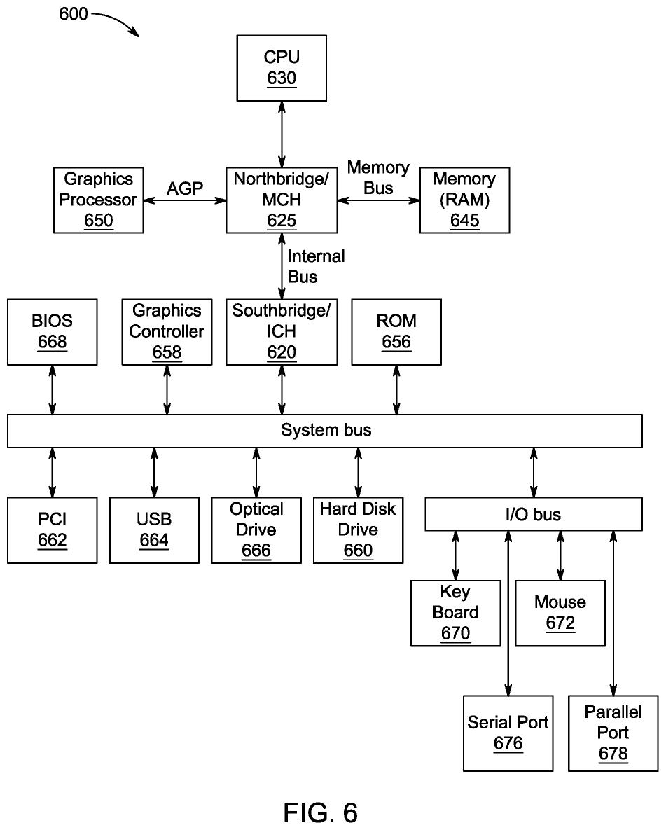

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 6.

FIG. 6 shows a schematic diagram of a data processing system, according to certain embodiments, for performing the functions of the exemplary embodiments. The data processing system is an example of a computer in which code or instructions implementing the processes of the illustrative embodiments may be located.

In FIG. 6, data processing system 600 employs a hub architecture including a north bridge and memory controller hub (NB/MCH) 625 and a south bridge and input/output (I/O) controller hub (SB/ICH) 620. The central processing unit (CPU) 630 is connected to NB/MCH 625. The NB/MCH 625 also connects to the memory 645 via a memory bus, and connects to the graphics processor 650 via an accelerated graphics port (AGP). The NB/MCH 625 also connects to the SB/ICH 620 via an internal bus (e.g., a unified media interface or a direct media interface). The CPU Processing unit 630 may contain one or more processors and even may be implemented using one or more heterogeneous processor systems.

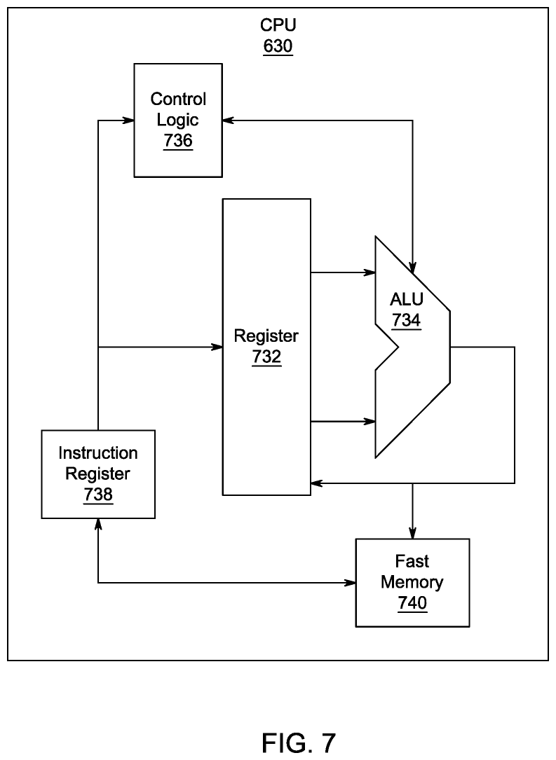

For example, FIG. 7 shows one implementation of CPU 630. In one implementation, the instruction register 738 retrieves instructions from the fast memory 740. At least part of these instructions are fetched from the instruction register 738 by the control logic 736 and interpreted according to the instruction set architecture of the CPU 630. Part of the instructions can also be directed to the register 732. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 734 that loads values from the register 732 and performs logical and mathematical operations on the loaded values according to the instructions. The results from these operations can be feedback into the register and/or stored in the fast memory 740. According to certain implementations, the instruction set architecture of the CPU 630 can use a reduced instruction set architecture, a complex instruction set architecture, a vector processor architecture, a very large instruction word architecture. Furthermore, the CPU 630 can be based on the Von Neuman model or the Harvard model. The CPU 630 can be a digital signal processor, an FPGA, an ASIC, a PLA, a PLD, or a CPLD. Further, the CPU 630 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 6, the data processing system 600 can include that the SB/ICH 620 is coupled through a system bus to an I/O Bus, a read only memory (ROM) 656, universal serial bus (USB) port 664, a flash binary input/output system (BIOS) 668, and a graphics controller 658. PCI/PCIe devices can also be coupled to SB/ICH 688 through a PCI bus 662.

The PCI devices may include, for example, Ethernet adapters, add-in cards, and PC cards for notebook computers. The Hard disk drive 660 and CD-ROM 666 can use, for example, an integrated drive electronics (IDE) or serial advanced technology attachment (SATA) interface. In one implementation the I/O bus can include a super I/O (SIO) device.

Further, the hard disk drive (HDD) 660 and optical drive 666 can also be coupled to the SB/ICH 620 through a system bus. In one implementation, a keyboard 670, a mouse 672, a parallel port 678, and a serial port 676 can be connected to the system bus through the I/O bus. Other peripherals and devices that can be connected to the SB/ICH 620 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

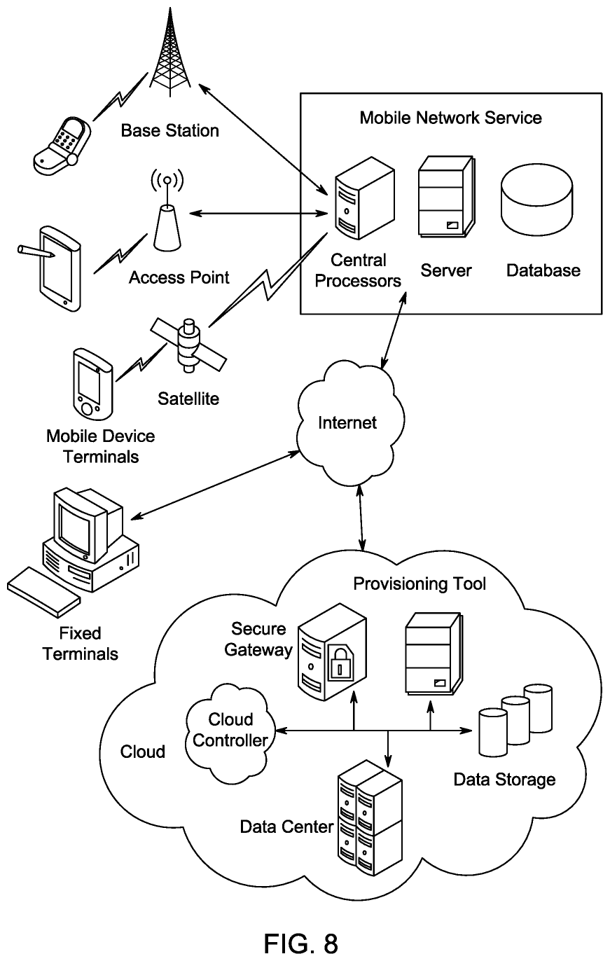

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 8, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Aspects of the present disclosure describe the apparatus 100 and the system 300. The applications of the system 300 may be multifaceted. In particular, the system 300 may be used during surgeries to monitor a fluid balance in order to prevent complications. The real-time data from the system 300 may guide fluid replacement strategies, consequently reducing the risk of post-operative complications. Further, in critical care settings, accurate fluid balance assessment is essential and the system 300 may offer continuous monitoring, aiding in rapid responses to fluid imbalances and optimizing patient care. Additionally, in a plurality of critical and deceptive illnesses, the ability of the system 300 to differentiate between vasodilation-related changes and fluid loss due to increase in vascular permeability helps the healthcare practitioners to tailor interventions more effectively. The system 300 provides individualized fluid needs estimations, enhancing personalized patient care and reducing the risk of over-hydration and under-hydration. In some aspects, the system 300 may have retrofitting abilities, in order to be retrofitted to existing medical equipment, to further increase the sustainability and economics of the system 300.

Numerous modifications and variations of the present disclosure are possible in light of the above teachings. It is therefore to be understood that within the scope of the appended claims, the invention may be practiced otherwise than as specifically described herein.

1. A fluid monitoring apparatus for a venous system of a human body, comprising:
a catheter tube insertable into a blood vessel of the venous system of the human body and configured to have fluid from the blood vessel enter and exit the catheter tube, wherein the catheter tube comprises at least one lumen;
at least four sensors attached to an internal surface of the catheter tube at a tip of the catheter tube and in contact with the fluid when inserted in the blood vessel, wherein the at least four sensors are disposed in tandem at the tip of the catheter tube and wherein the at least four different sensors are selected from the group consisting of:
a first pressure sensor attached at an insertable tip of the catheter tube;
a fluid movement sensor attached proximal to the insertable tip of the catheter tube;
a vasodilation detector attached at a first sector of a middle section of the catheter tube;
a second pressure sensor attached at a second sector of the middle section of the catheter tube; and
a volume sensor attached at a distal section of the catheter tube; and
a controller communicatively coupled to the at least four sensors.

2. The fluid monitoring apparatus of claim 1, wherein the controller further comprises a transducer in communication with the first pressure sensor and the second pressure sensor.

3. The fluid monitoring apparatus of claim 1, wherein each of the first pressure sensor and the second pressure sensor are at least one of a piezo-resistive sensor, photo-electric sensor and a photo-optic sensor, wherein the fluid movement sensor is at least one of an electromagnetic sensor and an electrochemical sensor, wherein the vasodilation detector is at least one of a capacitive strain gauge sensor or a bio-impedance sensor, and wherein the volume sensor is at least one of a photoplethysmography (PPG) sensor and an electromagnetic sensor.

4. The fluid monitoring apparatus of claim 1, wherein the controller is in communication with an external position sensor.

5. The fluid monitoring apparatus of claim 4, wherein the external position sensor is configured to be connected to an external surface of the human body.

6. The fluid monitoring apparatus of claim 4, further comprising an external cable connected between the external position sensor and the controller.

7. The fluid monitoring apparatus of claim 1, further comprising a plurality of cables connected between the at least four sensors attached to the catheter tube and the controller.

8. The fluid monitoring apparatus of claim 1, wherein each of the at least four sensors has a length in a range from 1 cm up to 3 cm.

9. The fluid monitoring apparatus of claim 1, wherein each of the at least four sensors has an external diameter in a range from 1 mm up to 2 mm.

10. The fluid monitoring apparatus of claim 1, wherein each of the at least four sensors has an external diameter up to 0.9 times of a diameter of the catheter tube.

11. The fluid monitoring apparatus of claim 1, wherein the at least four sensors are longitudinally spaced apart from one another inside the catheter tube, wherein a flexible separator is present between each sensor of the at least four sensors, wherein the flexible separator comprises a flexible cellular polymer.

12. The fluid monitoring apparatus of claim 1, wherein the catheter tube is made of a flexible material.

13. The fluid monitoring apparatus of claim 1, wherein the catheter tube comprises an insertion port having a length of at least 1 cm at the insertable tip of the catheter tube for insertion of a guidewire from a center point of the insertion port and along an edge of the at least four sensors attached to the catheter tube.

14. The fluid monitoring apparatus of claim 1, wherein the catheter tube is a central venous catheter.

15. The fluid monitoring apparatus of claim 1, wherein the catheter tube is integrated into an intra-aortic balloon pump.

16. A fluid management system for an invasive monitoring of a venous system of a human body, comprising:
a catheter tube insertable into a blood vessel of the venous system of the human body through a guidewire, wherein the catheter tube comprises at least one lumen, wherein fluid from the blood vessel enters and exits the catheter tube;
at least four sensors attached to an internal surface of the catheter tube and in contact with the fluid when inserted in the blood vessel, wherein the at least four sensors are selected from
a first pressure sensor at an insertable tip of the catheter tube configured to measure a pressure at the insertable tip of the catheter tube;
a fluid movement sensor proximal to the insertable tip of the catheter tube configured to measure a fluid movement rate of the fluid in contact with the fluid movement sensor;
a vasodilation detector at a first sector of a middle section of the catheter tube configured to measure a change in a diameter of the blood vessel for an assessment of at least one of a vasodilation or a vasoconstriction; and
a volume sensor at a distal section of the catheter tube configured to measure a change in a volume of the fluid in contact with the volume sensor;
a controller communicatively coupled to the at least four sensors configured to receive a measurement value from each of the at least four sensors and to assess a fluid requirement of the human body;
a plurality of cables extending from the catheter tube to the controller configured to conduct communication between the at least four sensors and the controller; and
an insertion port having a length of at least 1 cm at the insertable tip of the catheter tube for insertion of a guidewire from a center point of the insertion port and along an edge of the at least four sensors attached to the catheter tube.

17. The fluid management system of claim 16, wherein the controller further comprises a transducer in communication with the first pressure sensor.

18. The fluid management system of claim 16, further comprises
a second pressure sensor attached at a second sector of the middle section of the catheter tube and configured to measure and communicate a pressure of the fluid against a wall of the blood vessel to the controller.

19. The fluid management system of claim 16, wherein the controller is connected to an external position sensor through an external cable.

20. The fluid management system of claim 19, wherein the external position sensor is configured to be connected to an external surface of the human body and to detect and communicate a change in an orientation of the human body to the controller.
